(19) United States
(12) Patent Application Publication
(10) Pub. No.: US 2026/0087354 A1
(43) Pub. Date: Mar. 26, 2026

(54) UNDETECTABLE TEXT GENERATION TO ASSIST IN MEDICAL DECISION MAKING

(71) Applicant: NEC Laboratories America, Inc., Princeton, NJ (US)

(72) Inventors: Wei Cheng, Princeton Junction, NJ (US); Haifeng Chen, West Windsor, NJ (US)

(21) Appl. No.: 19/325,914

(22) Filed: Sep. 11, 2025

Related U.S. Application Data
(60) Provisional application No. 63/698,668, filed on Sep. 25, 2024.

(51) Int. Cl. G06N 3/09; G06F 40/40; G06N 3/0475; G16H 50/20

(52) U.S. Cl. CPC G06N 3/09; G06F 40/40; G06N 3/0475; G16H 50/20

(57) ABSTRACT

Methods and systems include fine-tuning a small language model (SLM) to determine a first probability distribution. Text is generated with a large language model (LLM), including modifying a second probability distribution of the LLM using the first probability distribution so that the text is human-like. A detector is trained, using the text, to determine whether input text is generated by a human or by a language model.

### RELATED APPLICATION INFORMATION

This application claims priority to U.S. Patent Application No. 63/698,668, filed on Sep. 25, 2024, incorporated herein by reference in its entirety.

### BACKGROUND

### Technical Field

The present invention relates to large language models and, more particularly to avoiding detection of automatically generated text.

### Description of the Related Art

Large language models (LLMs) are effective at generating text which is difficult to distinguish from text written by a human being. An LLM may be instructed to generate text in a variety of styles. However, there are efforts to build tools which detect such automatically generated text. Existing detection methods include watermarking and fine-tuning classifiers directed toward a particular known model. These tools struggle with reliability and robustness, particularly as language models become larger and more sophisticated. However, detection tools are becoming more sophisticated as well.

### SUMMARY

A method includes fine-tuning a small language model (SLM) to determine a first probability distribution. Text is generated with a large language model (LLM), including modifying a second probability distribution of the LLM using the first probability distribution so that the text is human-like. A detector is trained, using the text, to determine whether input text is generated by a human or by a language model.

A system includes a hardware processor and a memory that stores a computer program. When executed by the hardware processor, the computer program causes the hardware processor to fine-tune an SLM to determine a first probability distribution, to generate text with an LLM, including modification of second probability distribution of the LLM using the first probability distribution so that the text is human-like, and to train a detector, using the text, to determine whether input text is generated by a human or by a language model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustrating the generation of undetectable human-like text, in accordance with an embodiment of the present invention;

FIG. 2 is a block/flow diagram of a method for generating and using human-like text that is difficult to detect, in accordance with an embodiment of the present invention;

FIG. 3 is a block diagram of a healthcare facility where text detection, trained by human-like generated text, is used to assist in medical decision making, in accordance with an embodiment of the present invention;

FIG. 4 is a block diagram of a computing device that can generate human-like text, in accordance with an embodiment of the present invention;

FIG. 5 is a diagram of an exemplary neural network architecture that can be used to implement part of a small language model, in accordance with an embodiment of the present invention; and

FIG. 6 is a diagram of an exemplary neural network architecture that can be used to implement part of a small language model, in accordance with an embodiment of the present invention.

### DETAILED DESCRIPTION OF PREFERRED EMBODIMENTS

To avoid detection of automatically generated text, a helper model may be used to guide and enhance the output of a large language model (LLM). A source model is contaminated by aligning its distribution to resemble human-written text using a fine-tuned, humanized small language model (SLM). This is equivalent to attacking a large model. Detectors are consistently fooled by proxy-attacked source models in both white-box and black-box settings. Detectors can be misled by a humanized SLM trained on cross-domain data sources. This approach evades detection while maintaining the quality of the output text.

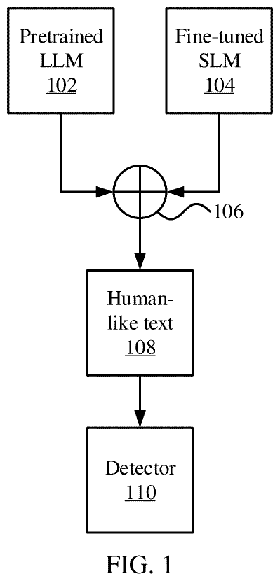

Referring now to FIG. 1, the generation of undetectable text is shown. Output from a pretrained LLM 102 is combined 106 with output from a fine-tuned SLM 104 to create human-like text 108. A detector 110 is configured to detect text that has been generated by the pre-trained LLM 102, but when it reviews the human-like text 108, it incorrectly determines that the human-like text 108 was written by a human being.

The LLM 102 and the SLM 104 differ in the number of parameters that they employ. For example, an LLM 102 may have a tens to hundreds of billions of parameters, whereas an SLM 104 may have less than ten billion parameters. The large number of parameters in the LLM 102 make it difficult to fine-tune those large models directly, but the smaller number of parameters in the SLM 104 make fine-tuning much more tractable. As the state of the art advances, the number of parameters included in an LLM and in an SLM may change, but the relative difficulty of fine-tuning such models will remain a concern.

Given a set of prompts χ and responses [symbol] , an auto-regessive model generates an output sentence y=[y₁, . . . , y_{T}]∈ [symbol] conditional on a prompt x∈χ, based on conditional probability distributions π(yₜ|x, y_{<t}), where each yₜ is a single token. Machine generative processes are indicated herein as M and human generative processes are indicated herein as H. The corresponding conditional probabilities are π_{M} and π_{H} respectively. An overall distribution of output text y for a given prompt x is expressed as:

$$ℙ ( y \vert x ) = {\prod }_{1}^{T} π ( {y}_{t} \vert x , {y}_{< t} )$$

which, for simplicity, is denoted by π(y|x).

Given a prompt-response pair (x, y), a detector D is essentially a binary classifier, with the task of detecting whether the response is generated from a known language model M or a human process H. The detector 110 is assumed to use an implicit reward function r(x, y) for its decisions, which gives higher reward for human-like texts compared to machine-generated texts.

The present embodiments use generative process M′, such that the detector 110 is unable to distinguish texts generated by M′ from those by H. This may be formulated as achieving an expected reward

$${𝔼}_{y ∼ {π}_{M} ′ ( \cdot \vert x )} r ( x , y )$$

on par with the human expected reward, given that the initial expected reward for M^{ref} is much smaller in comparison.

Preference-based reinforcement learning (PBRL) leverages human or evaluative feedback to optimize a model's behavior using reinforcement learning. To finetune a pre-trained language model M^{ref}, a preference dataset [symbol] :={(x, y^{w}, y^{l})} is used, where the responses y^{w}, y^{l}˜π^{ref}(⋅|x) are sampled from a reference policy π^{ref} that could be obtained after supervised fine-tuning (SFT), while preferences y^{w} [symbol] y^{l}|x are labeled either by an artificial intelligence (AI) system or human annotator, indicating y^{w} is preferred over y^{l} given the query x. In PBRL, the preference is assumed to be associated with a latent reward function r*. To learn this reward from the dataset, a Bradley-Terry model may be used, which assumes that the probability of y^{w} [symbol] y^{l}|x satisfies the following:

$$p ( {y}^{w} ≻ {y}^{l} \vert x ) := \frac{exp ( {r}^{*} ( x , {y}^{w} ) )}{exp ( {r}^{*} ( x , {y}^{w} ) ) + exp ( {r}^{*} ( x , {y}^{l} ) )}$$

It follows that the maximum-likelihood reward learning objective is

$${r}^{*} ← {arg max}_{r ∈ ℛ} {𝔼}_{( x , {y}^{w} , {y}^{l} ) ∼ 𝒟} [ log σ ( r ( x , {y}^{w} ) - r ( x , {y}^{l} ) ) ] ,$$

where σ is the sigmoid function. After obtaining the reward r*, the RL fine-tuning of a language model follows the objective:

$${π}^{*} ← {arg max}_{π} {𝔼}_{x ∼ 𝒟 , y ∼ π ( \cdot \vert x )} [ {r}^{*} ( x , y ) - {β𝔻}_{KL} [ π ( y \vert x ) \vert \vert {π}^{ref} ( y \vert x ) ] ]$$

Direct Preference Optimization (DPO) provides a solution for π* without learning the reward function, by optimizing the objective

$${π}^{*} ← {arg max}_{π} {𝔼}_{( x , {y}^{w} , {y}^{l} ) ∼ D} [ log σ ( β log \frac{π ( {y}^{w} \vert x )}{{π}^{ref} ( {y}^{w} \vert x )} - β log \frac{π ( {y}^{l} \vert x )}{{π}^{ref} ( {y}^{l} \vert x )} ) ]$$

where [symbol] is a set of reward functions.

There is a significant computational cost to using PBRL to fine-tune LLMs for detector evasion, due to the large size of the models (e.g., 70 B parameters). Directly fine-tuning such large models for attacks is impractical. The present embodiments use DPO to fine-tune an SLM towards an optimal reward until it reaches the same level of reward for the human process according to a scoring detector. The LLM is adapted to achieve the same expected reward.

DPO fine-tuning can be applied for bypassing detectors. For each prompt x∈χ in the dataset, sample response pairs (y₁, y₂) are generated by the reference model π^{ref} To obtain the dataset [symbol] ={(x, y^{w}, y^{l})}, preference labels are assigned by comparing a scoring detector's human-ness score s(x, y) on the responses: if s(x, y₁)>s(x, y₂), assign preference label y₁ [symbol] y₂ and let y^{w}=y₁, y^{l}=y₂; otherwise assign y^{w}=y₂, y^{l}=y₁. The generated dataset [symbol] is then used to fine-tune a pre-trained SLM

$${M}_{s}^{ref}$$

with DPO. This produces a humanized SLM denoted as Mₛ as the proxy attacker. This label assignment process can be approximated by the Bradley-Terry model when r(x,y)=C·s(x, y) with a large constant C. It is therefore assumed that the detector 110 follows an implicit reward function r to generate [symbol] .

Generally, for fine-tuning a language model using DPO with preference data from detectors, given a starting reference model M^{ref} with a low reward, there exists hyperparameter β such that the optimal model M* fine-tuned on the DPO objective achieves the same expected reward as H:

$${𝔼}_{x ∼ 𝒟 , y ∼ {π}_{M} * ( \cdot \vert x )} r ( x , y ) = {𝔼}_{x ∼ 𝒟 , y ∼ {π}_{H} ( \cdot \vert x )} r ( x , y ) .$$

Intuitively, this result is due to the effect of β on the fine-tuned model: the smaller β is, the closer M* approaches optimal reward, while larger β results in higher similarity to the reference model and hence higher quality. This is in line with the RL objective, in which the β term controls the strength of regularization.

With a humanized SLM trained on the DPO objective, the LLM's next-token output distribution is altered by multiplying a logit offset for each token probability. This offset is calculated as the ratio between the logits of the proxy-attacker small model Mₛ and those of the pre-trained reference small model

$${M}_{s}^{ref}$$

Formally, at each time step t, given the tokens y_{<t}, the probability distribution of our proxy-attacker large model M′ is calculated as

$${π}_{{M}^{′}} ( {y}_{t} \vert x , {y}_{< t} ) = \frac{1}{{Z}_{x , y < t}} {π}_{M}^{ref} ( {y}_{t} \vert x , {y}_{< t} ) {( \frac{{π}_{{M}_{z}} ( {y}_{t} \vert x , {y}_{< t} )}{{π}_{{M}_{s}}^{ref} ( {y}_{t} \vert x , {y}_{< t} )} )}^{α}$$

where

$${Z}_{x , y < t} = {\sum }_{{y}_{t}} {π}_{M}^{re f} ( {y}_{t} \vert x , {y}_{< t} ) {( \frac{{π}_{{M}_{s}} ( {y}_{t} \vert x , {y}_{< t} )}{{π}_{{M}_{s}}^{ref} ( {y}_{t} \vert x , {y}_{< t} )} )}^{α}$$

is the normalization factor and α is the attack ratio. The term

$${( \frac{{π}_{{M}_{s}} ( {y}_{t} \vert x , {y}_{< t} )}{{π}_{{M}_{s}}^{{r}_{s} t} ( {y}_{t} \vert x , {y}_{< t} )} )}^{α}$$

captures the distribution shift from pre-trained to fine-tuned on the small model, and attempts to approximate the corresponding shift on the large model

$$\frac{{π}_{{MM}^{′}} ( {y}_{t} \vert x , {y}_{< t} )}{{π}_{M}^{{E}^{′ t}} ( {y}_{t} \vert x , {y}_{< t} )} .$$

Using the logarithm of logits allows us to derive the probability distribution from the proxy-attacked model M′ as

$${p}_{{M}^{′}} ( {y}_{t} \vert x , {y}_{< t} ) = softmax [ {p}_{M}^{ref} ( {y}_{t} \vert x , {y}_{< t} ) + α ( {p}_{{M}_{s}} ( {y}_{t} \vert x , {y}_{< t} ) - {p}_{{M}_{s}}^{ref} ( {y}_{t} \vert x , {y}_{< t} ) ) ]$$

where

$${p}_{M}^{ref} , {p}_{{M}_{s}} and {p}_{{M}_{s}}^{ref}$$

are the logarithmic logits for the pre-trained large model M^{ref}, the finetuned small model Mₛ and the pre-trained small model

$${M}_{s}^{ref}$$

respectively. M and Mₛ only need to share the same vocabulary. Compared to generically fine-tuning the large model using DPO, we have the following theorem, assuming the small fine-tuned model Mₛ achieves an optimum according to the DPO objective, with β=β₀, the inference model M′ is the same as an alternative large model fine-tuned on the DPO objective with β=β/α. The attack ratio a has a similar, but inverted, effect as β on the resulting model M′, where larger values of a lead to higher reward and better detection evasion, while smaller values of α keep M′ closer to the reference model M^{ref}. The attack ratio a effectively controls the trade-off between evasion performance and quality at the decoding phase, in contrast to β₀, which is applied at fine-tuning.

In addition, given parameter β₀ for fine-tuning the SLM Mₛ on the DPO objective, there exists an attack ratio α>0 such that the resulting proxy attacker M′ achieves the same expected reward as the human process H according to the detector D, thereby evading detection.

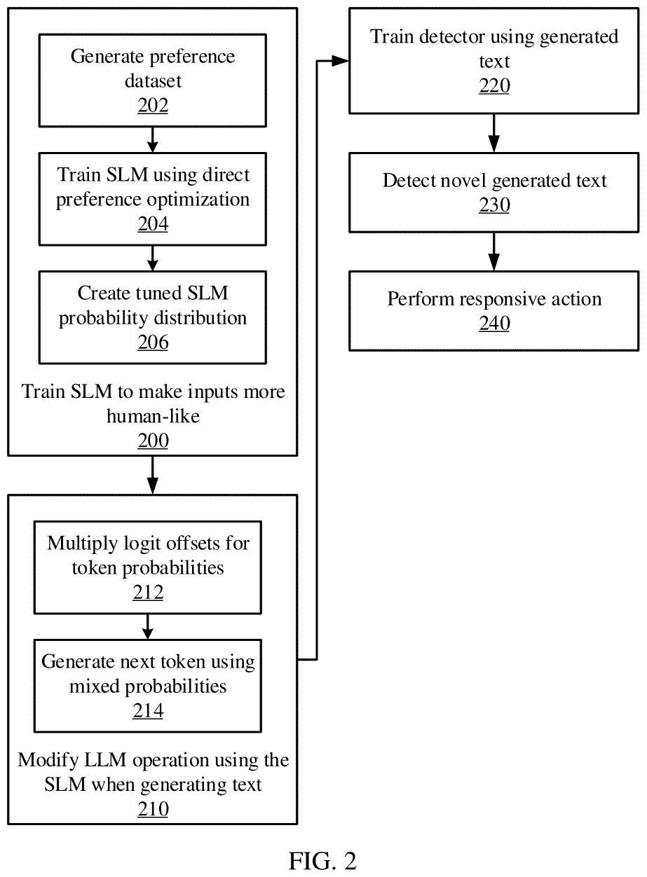

Referring now to FIG. 2, a method of training and using an AI-generated text detector is shown. Block 200 trains an SLM 104 to make inputs more human-like. Fine-tuning the SLM includes generating a preference dataset in block 202, comparing pairs of LLM outputs that are generated responsive to a shared prompt. The dataset includes an indication of preferences, for example set by a human annotator, providing ground truth information as to which of the pair is more human-like. Block 204 performs direct preference optimization to train the SLM, such that block 206 creates a tuned SLM probability distribution π* as described above.

Block 210 modifies operation of the pretrained LLM 102 using the fine-tuned SLM 104 when the LLM 102 is used to generate text. In particular, block 212 multiplies logit offsets for next-token probabilities using the fine-tuned SLM probability distribution, with a distribution shift factor that captures the distribution shift from the pre-trained SLM to the fine-tuned SLM. Block 214 then generates a next token in a sequence using these mixed probabilities from the pre-trained LLM 102 and the fine-tuned SLM 104 to create text that appears more human than would the LLM's default output.

The generated text may be used in block 220 to help train a detector 110 to better discriminate between human-generated text and LLM-generated text. Because block 210 produces more human-like text, it can be used to train the detector 110 to identify text that has been altered to disguise its AI origins.

Block 230 may then use the trained detector to identify novel examples AI-generated text. For example, AI-generated text may be provided as an input to a form in a medical context, describing a patient's symptoms. Detecting AI-generated text is cause for caution in such a situation, as it indicates that the description may include hallucinations or may be fabricated entirely. Block 240 then performs an action that responds to the detection of the AI-generated text. Thus the detector 110, which may be trained through the use of automatically generated human-like text, may be used to aid in medical decision making. In another embodiment, the trained detector may be used by a patient to determine whether they are interacting with a human doctor or with an AI chatbot, to help the patient trust the medical advice that they receive.

In some embodiments, the human-like text can be used directly in a medical context. For example, some individuals' natural writing style can be mistaken for AI-generated text, due to their use of stylistic and semantic patterns that are commonly used by LLMs. Block 210 may therefore generate text on behalf of the individual to make their responses to medical questions more likely to be accepted as genuine. The responsive action 240 may therefore perform responsive actions based on accurate information presented in the human-like generated text, rather than raising misleading concerns about AI-generated text.

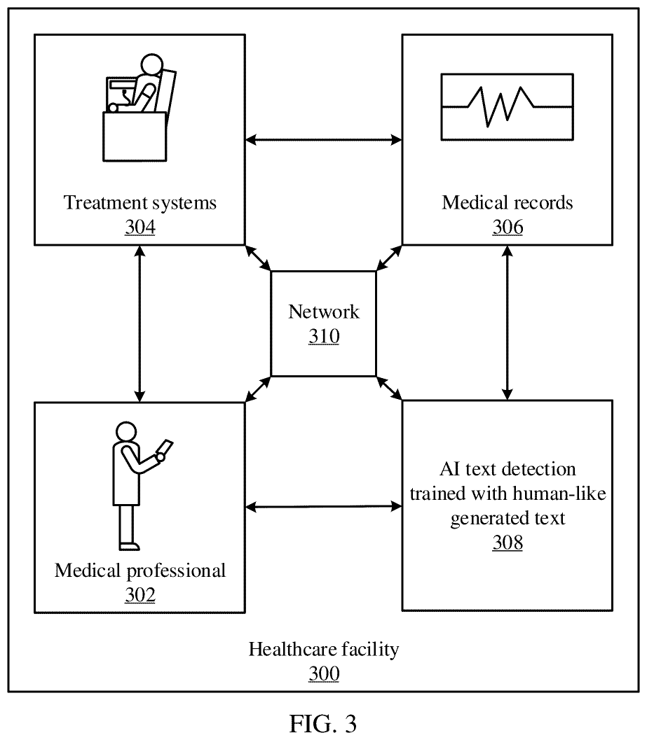

Referring now to FIG. 3, a diagram of AI-generated text detection is shown in the context of a healthcare facility 300. AI text detection trained with human-like generated text 308 may be used to identify text that has been created by an AI language model, for example including information present in a patient's medical records 306.

The healthcare facility may include one or more medical professionals 302 who review information extracted from a patient's medical records 306 to determine their healthcare and treatment needs. These medical records 306 may include self-reported information from the patient, test results, and notes by healthcare personnel made to the patient's file. Treatment systems 304 may furthermore monitor patient status to generate medical records 306 and may be designed to automatically administer and adjust treatments as needed.

AI text detection trained with human-like generated text 308 may be used to determine that medical information has been generated by an AI model rather than by a patient or medical professional. This condition indicates a hazard to the patient, as it indicates that the submitted information is potentially unreliable due to, e.g., hallucinations and fabrications. Medical professionals 302 may then make medical decisions about patient healthcare suited to the patient's needs, taking into account the fact that some of the available information may be suspect. For example, the medical professionals 302 may discount AI-generated text when making treatment decisions and may prescribe particular medications, surgeries, and/or therapies that are appropriate to the diagnosis disease.

The different elements of the healthcare facility 300 may communicate with one another via a network 310, for example using any appropriate wired or wireless communications protocol and medium. Thus AI text detection trained with human-like generated text 308 receives data from medical professionals 302 and from medical records 306, and may identify which inputs are human-generated and which are potentially unreliable AI-generated text. AI text detection trained with human-like generated text 308 may further coordinate with treatment systems 304 in some cases to automatically administer or alter a treatment. For example, if a treatment is based on AI-generated information about the patient, the AI text detection trained with human-like generated text 308 may automatically trigger halting the administration of a medication.

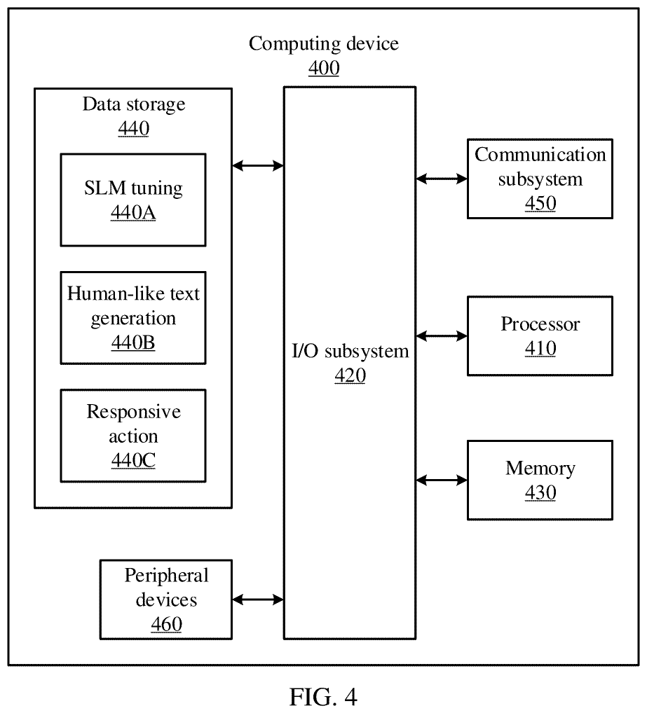

Referring now to FIG. 4, an exemplary computing device 400 is shown, in accordance with an embodiment of the present invention. The computing device 400 is configured to perform visual question answering.

The computing device 400 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 400 may be embodied as one or more compute sleds, memory sleds, or other racks, sleds, computing chassis, or other components of a physically disaggregated computing device.

As shown in FIG. 4, the computing device 400 illustratively includes the processor 410, an input/output subsystem 420, a memory 430, a data storage device 440, and a communication subsystem 450, and/or other components and devices commonly found in a server or similar computing device. The computing device 400 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 430, or portions thereof, may be incorporated in the processor 410 in some embodiments.

The processor 410 may be embodied as any type of processor capable of performing the functions described herein. The processor 410 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 430 may be embodied as any type of volatile or non-volatile memory or data storage capable of performing the functions described herein. In operation, the memory 430 may store various data and software used during operation of the computing device 400, such as operating systems, applications, programs, libraries, and drivers. The memory 430 is communicatively coupled to the processor 410 via the I/O subsystem 420, which may be embodied as circuitry and/or components to facilitate input/output operations with the processor 410, the memory 430, and other components of the computing device 400. For example, the I/O subsystem 420 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 420 may form a portion of a system-on-a-chip (SOC) and be incorporated, along with the processor 410, the memory 430, and other components of the computing device 400, on a single integrated circuit chip.

The data storage device 440 may be embodied as any type of device or devices configured for short-term or long-term storage of data such as, for example, memory devices and circuits, memory cards, hard disk drives, solid state drives, or other data storage devices. The data storage device 440 can store program code 440A for fine-tuning the SLM, 440B for generating human-like text, and/or 440° C. for performing responsive actions. Any or all of these program code blocks may be included in a given computing system. The communication subsystem 450 of the computing device 400 may be embodied as any network interface controller or other communication circuit, device, or collection thereof, capable of enabling communications between the computing device 400 and other remote devices over a network. The communication subsystem 450 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 400 may also include one or more peripheral devices 460. The peripheral devices 460 may include any number of additional input/output devices, interface devices, and/or other peripheral devices. For example, in some embodiments, the peripheral devices 460 may include a display, touch screen, graphics circuitry, keyboard, mouse, speaker system, microphone, network interface, and/or other input/output devices, interface devices, and/or peripheral devices.

Of course, the computing device 400 may also include other elements (not shown), as readily contemplated by one of skill in the art, as well as omit certain elements. For example, various other sensors, input devices, and/or output devices can be included in computing device 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 400 are readily contemplated by one of ordinary skill in the art given the teachings of the present invention provided herein.

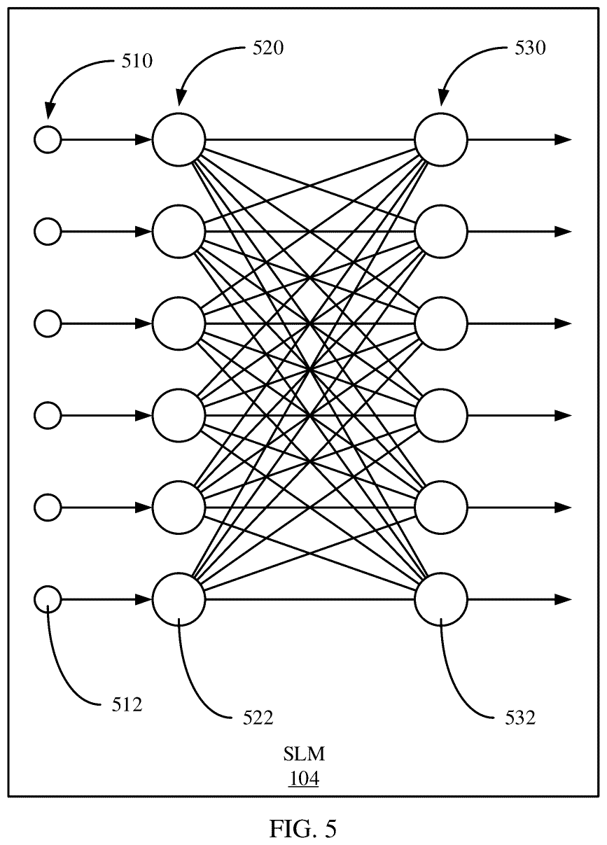

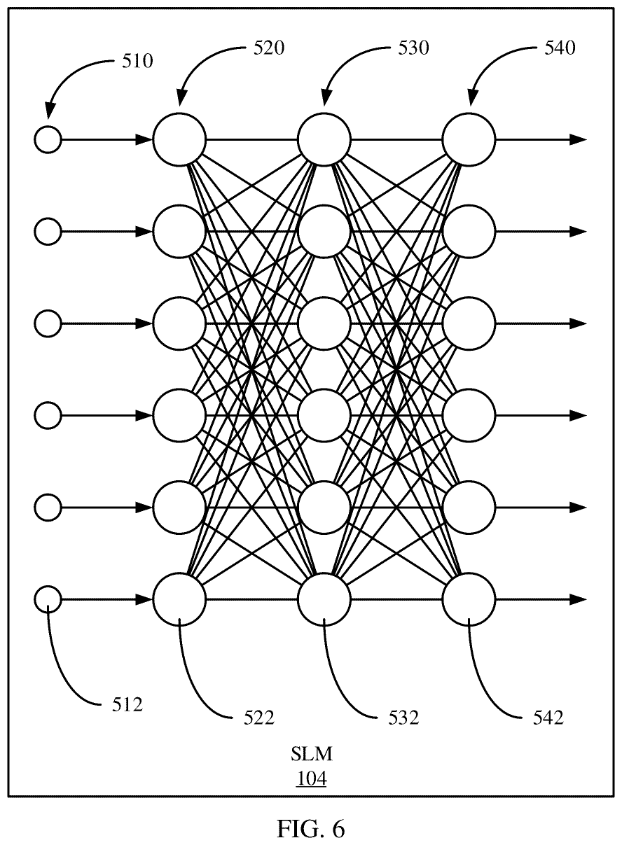

Referring now to FIGS. 5 and 6, exemplary neural network architectures are shown, which may be used to implement parts of the present machine learning models, such as the SLM 104. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 520 of source nodes 522, and a single computation layer 530 having one or more computation nodes 532 that also act as output nodes, where there is a single computation node 532 for each possible category into which the input example could be classified. An input layer 520 can have a number of source nodes 522 equal to the number of data values 512 in the input data 510. The data values 512 in the input data 510 can be represented as a column vector. Each computation node 532 in the computation layer 530 generates a linear combination of weighted values from the input data 510 fed into input nodes 520, and applies a non-linear activation function that is differentiable to the sum. The exemplary simple neural network can perform classification on linearly separable examples (e.g., patterns).

A deep neural network, such as a multilayer perceptron, can have an input layer 520 of source nodes 522, one or more computation layer(s) 530 having one or more computation nodes 532, and an output layer 540, where there is a single output node 542 for each possible category into which the input example could be classified. An input layer 520 can have a number of source nodes 522 equal to the number of data values 512 in the input data 510. The computation nodes 532 in the computation layer(s) 530 can also be referred to as hidden layers, because they are between the source nodes 522 and output node(s) 542 and are not directly observed. Each node 532, 542 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 532 in the one or more computation (hidden) layer(s) 530 perform a nonlinear transformation on the input data 512 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
fine-tuning a small language model (SLM) to determine a first probability distribution;
generating text with a large language model (LLM), including modifying a second probability distribution of the LLM using the first probability distribution so that the text is human-like; and
training a detector, using the text, to determine whether input text is generated by a human or by a language model.

2. The method of claim 1, wherein modifying the second probability distribution includes multiplying the second probability distribution by a distribution shift factor that captures a distribution shift to the fine-tuned SLM from the SLM prior to fine-tuning.

3. The method of claim 2, wherein the distribution factor is a ratio of the first probability distribution and a pretrained reference probability distribution of the SLM.

4. The method of claim 1, wherein fine-tuning the SLM includes performing direct preference optimization using a preference dataset that includes pairs of generated text.

5. The method of claim 4, wherein the preference dataset includes labels for the pairs of generated text that indicate which of each pair is preferred.

6. The method of claim 1, wherein generating text includes generating a next token using the modified second probability distribution.

7. The method of claim 1, further comprising using the detector to determine that a novel input text was generated by a language model and performing an action responsive to the novel input text.

8. The method of claim 7, wherein the novel input text is a description of a patient's medical condition and wherein the detector is used to assist in medical decision making.

9. The method of claim 8, wherein the action includes halting a treatment responsive to a determination that the novel input text is unreliable due to having been generated by the language model.

10. The method of claim 1, wherein the LLM and the SLM are both machine learning models, with the LLM having more parameters than the SLM.

11. A system, comprising:
a hardware processor; and
a memory that stores a computer program which, when executed by the hardware processor, causes the hardware processor to:
fine-tune a small language model (SLM) to determine a first probability distribution;
generate text with a large language model (LLM), including modification of second probability distribution of the LLM using the first probability distribution so that the text is human-like; and
train a detector, using the text, to determine whether input text is generated by a human or by a language model.

12. The system of claim 11, wherein modification of the second probability distribution includes multiplication of the second probability distribution by a distribution shift factor that captures a distribution shift to the fine-tuned SLM from the SLM prior to fine-tuning.

13. The system of claim 12, wherein the distribution factor is a ratio of the first probability distribution and a pretrained reference probability distribution of the SLM.

14. The system of claim 11, wherein the fine-tuning of the SLM includes direct preference optimization using a preference dataset that includes pairs of generated text.

15. The system of claim 14, wherein the preference dataset includes labels for the pairs of generated text that indicate which of each pair is preferred.

16. The system of claim 11, wherein generation of text includes a next token using the modified second probability distribution.

17. The system of claim 11, wherein the computer further causes the hardware processor to use the detector to determine that a novel input text was generated by a language model and to perform an action responsive to the novel input text.

18. The system of claim 17, wherein the novel input text is a description of a patient's medical condition and wherein the detector is used to assist in medical decision making.

19. The system of claim 18, wherein the action includes halting a treatment responsive to a determination that the novel input text is unreliable due to having been generated by the language model.

20. The system of claim 11, wherein the LLM and the SLM are both machine learning models, with the LLM having more parameters than the SLM.
